(19) United States
(12) Patent Application Publication
(10) Pub. No.: US 2023/0137262 A1
(43) Pub. Date: May 4, 2023

(54) LIFTING THREAD FOR PRESS

(71) Applicant: Feel Korea Co., Ltd., Gwangju (KR)

(72) Inventor: DONG JIN KIM, Gwangju (KR)

(73) Assignee: Feel Korea Co., Ltd., Gwangju (KR)

(21) Appl. No.: 17/973,543

(22) Filed: Oct. 26, 2022

(30) Foreign Application Priority Data
Nov. 4, 2021 (KR) 10-2021-0150601

(51) Int. Cl. A61B 17/06; A61F 2/00; B29D 99/00

(52) U.S. Cl. CPC A61B 17/06166; A61F 2/0059; B29D 99/0078; A61B 2017/06176; A61B 2017/00526

(57) ABSTRACT

Disclosed is a lifting thread for a press. A thread in which protrusions of a protruding structure, evenly distributed along a surface portion of a yarn, are configured through a pressing method of a press device is provided. Accordingly, even though a tensile force and a traction force of the entire thread have a significant difference depending on the hardness and the degree of density of the skin tissue lipids of patients to be treated, it is possible to provide a customized and molded lifting thread.

### BACKGROUND

### Technical Field

Various embodiments generally relate to a lifting thread for a press, and more particularly, to a lifting thread for a press, which is formed with protrusion structures capable of being hooked, in a manner of pressing portions of the thread while a press is rotated around the outer surface of the thread.

### Related Art

In general, a suture thread used in surgery in a manner of suturing the incised skin has been expanded to the fields of plastic surgery and beauty as time goes by, and has been rapidly developed as the structural shape thereof is changed and the material thereof is also changed together.

In particular, in the field of plastic surgery and beauty, according to the change of the structural shape of a yarn and the change of materials, yarns for a lifting use are frequently used and utilized. Such yarns for a lifting use lift the sagging or a wrinkled portion of the skin in such a manner that, in the process in which the yarns are generally inserted in one direction of the skin, a pulling force according to tension acts in a direction opposite to the insertion.

However, in most of the existing yarns used for a lifting use, since protrusions are formed in a linear arrangement structure on the surface of a yarn manufactured in a cutting method, when a pulling force acts in the direction opposite to the insertion in the process in which the yarn is inserted into the skin, all of the pulling force is concentrated on the protrusions which are formed in the linear arrangement structure.

As, in this way, all of the pulling force is concentrated on the protrusions which are formed in the linear arrangement structure, it is a frequent occurrence that the protrusions do not withstand the concentrated pulling force to be bent or cut portions of the protrusions are torn and separated away.

When, in this way, the protrusions are formed in the cutting method, a problem may arise in that the size and thickness of the protrusions cannot help but be limited, and it is also pointed out as a problem that the tips of the protrusions are markedly sharp to overly stimulate the surrounding tissues or nerves of the skin.

Moreover, due to damage to the yarn according to the existing cutting method, a problem may arise in that the strength of the yarn may significantly decrease compared to other yarns of the same size, and it is also pointed out as a problem that, since the thin protrusions manufactured by the cutting method have rapid bioabsorbability in the body, they may easily lose their holding force for the tissues of the skin.

While lifting threads having a knotted structure are also widely distributed, knots may cause damage to tissues and nerves when the lifting threads are inserted into the subcutaneous tissues, and actually, it is a frequent occurrence that a patient suffers from severe pain of a sequela due to the presence of knotted portions.

While, as described above, various methods have been used for the manufacture of medical threads having a tissue holding function, the continuous development of technology that may make up for the shortcomings of the current products and may be immediately commercialized to be practically used is currently demanded.

### PRIOR ART DOCUMENT

### Patent Document

### SUMMARY

Various embodiments are directed to a lifting thread for a press, which has protrusions on the surface thereof to be effectively used in a surgical procedure and is able to be utilized for a customized lifting purpose according to the lipid hardness and degree of density of the skin.

In an embodiment, a lifting thread for a press may be provided, wherein the lifting thread is a thread in which protrusions of a protruding structure, evenly distributed along a surface portion of a yarn, are configured through a pressing method of a press device.

The protrusions may be formed by a rotary press which presses a surface portion of the yarn at regular intervals while being rotated around a rear portion of the yarn spread in a predetermined straight length through an appropriate tension inside the press device.

The protrusions may be formed in a pressing method through heating and cooling processing according to rotation and pressing operations of the rotary press.

The yarn may be made of at least any one substance among PDO, PLLA and PCL.

A pressing space for a mold of the rotary press may be 70 to 90 μm.

In the lifting thread for a press according to the embodiments of the present disclosure, in a process in which the lifting thread for a press is inserted into the skin to improve sagging or wrinkles of the skin, even when a pulling force is applied in a direction opposite to the insertion, through the effect of dispersing the concentration of the pulling force due to the structure of protrusions evenly distributed on the surface of the lifting thread, it is possible to anticipate an effect of preventing a phenomenon in which the protrusions are torn or separated from the lifting thread.

In addition, in the lifting thread for a press according to the embodiments of the present disclosure, since the protrusions are formed through a rotary press, mechanical processing for the size and thickness of the protrusions may be adjusted, and thus, it is possible to eliminate limitations on the size and thickness of the protrusions.

Moreover, in the lifting thread for a press according to the embodiments of the present disclosure, since the ends of the protrusions may be processed into a blunt structure through the adjustment in the mechanical processing, unnecessary stimulation to the surrounding tissues and nerves of the skin may be significantly reduced. As a result, this may achieve effects in which the occurrence of damage to tissues and nerves of the skin remarkably decreases and the degree of pain experienced by a patient when the thread is lifted in the skin may be significantly reduced.

Also, in the lifting thread for a press according to the embodiments of the present disclosure, since the thickness of the body of the thread may be kept constant compared to the existing cutting thread, it is possible to achieve an effect that the strength of the lifting thread may be continuously maintained even after skin lifting.

Further, in the lifting thread for a press according to the embodiments of the present disclosure, since the strength of yarns is larger than the other existing yarns of the same size and even the bioabsorbability of the protrusions in the body is appropriate, it is possible to achieve an effect that a holding force for the tissues of the skin is ensured.

### BRIEF DESCRIPTION OF THE DRAWINGS

FIG. 1 is a view three-dimensionally showing the structure of a lifting thread for a press in accordance with a first embodiment of the present disclosure.

FIG. 2 is a longitudinal sectional view of the lifting thread for a press shown in FIG. 1.

FIG. 3 is a longitudinal sectional view of a lifting thread for a press in accordance with a second embodiment of the present disclosure.

FIG. 4 is a longitudinal sectional view of a lifting thread for a press in accordance with a third embodiment of the present disclosure.

FIG. 5 is a longitudinal sectional view of a lifting thread for a press in accordance with a fourth embodiment of the present disclosure.

FIG. 6 is a detailed longitudinal sectional view of the lifting thread for a press in accordance with the fourth embodiment of the present disclosure.

FIG. 7 is a view showing as an example the cross-section of a lifting thread for a press in accordance with the present disclosure.

### DETAILED DESCRIPTION

The present disclosure should be interpreted in a manner that includes the scope of rights reaching the technical spirit through various modified embodiments. The exemplary embodiments are provided for making the prevent disclosure complete and for fully conveying the scope of the present disclosure to those skilled in the art to which the present disclosure pertains. It is to be noted that the scope of the present disclosure is defined only by the claims.

Moreover, the drawings attached to the present disclosure serve as a way to facilitate the understanding of the description of the present disclosure. Therefore, the drawings attached to the present disclosure do not represent the technical spirit of the present disclosure, and the technical spirit of the present disclosure should not be understood as being limitedly interpreted by the attached drawings.

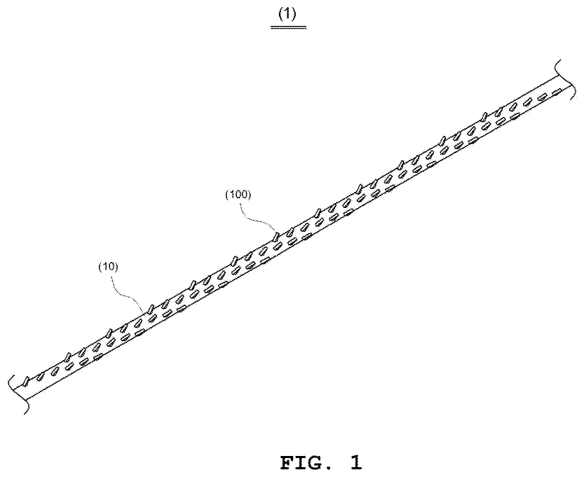

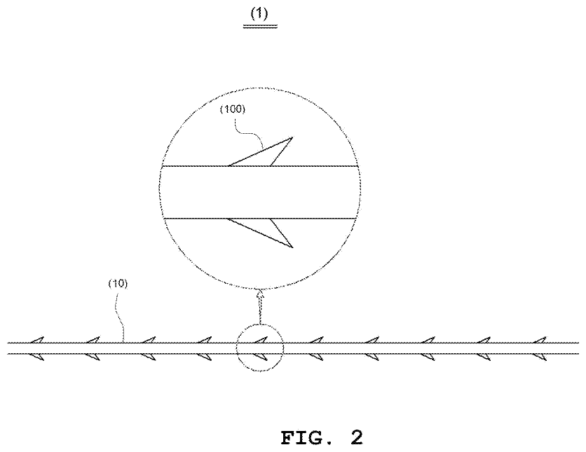

In a lifting thread for a press in accordance with a first embodiment of the present disclosure, for example, as shown in FIGS. 1 and 2, by heating and cooling the raw material of a yarn (through quick heating and quick cooling) under a specific temperature and pressure condition and then performing pressing, protrusions 100 may be formed on a yarn 10 which forms the whole body of a thread (insertion thread) 1.

The protrusions 100 formed on the yarn 10 which forms the whole body of the thread (insertion thread) 1 may be formed through a rotary press of a pressing device. For example, the rotary press is rotated along the length of the yarn 10 which is spread in a straight line state in the pressing device while maintaining a tension, and performs pressing at regular intervals on the surface of the yarn 10.

The yarn 10, which forms the lifting thread (insertion thread) 1 used in the fields of plastic surgery and beauty to improve sagging or wrinkles of the skin, may be made of a material as a polymer substance, for example, a substance such as PDO, PLLA and PCL, and may be based on the molecular orientation and thermal properties of a raw material.

Since the lifting thread (insertion thread) 1 has a directional molecular orientation therein, a high tensile strength of a polymer may be maintained.

When a polymer is heated in a range from a melting point to 40° C. below the melting point (Tm to Tm−40° C.), ductility increases, but a shrinkage deformation occurs to lose a molecular orientation, and a tensile strength required for lifting according to insertion into the skin is also lost.

However, as a polymer is fixed at both ends and heat is applied in the same range so that a shrinkage deformation does not occur in the polymer even by such heating, the lifting thread (insertion thread) 1 made of a polymer has characteristics that ductility increases and a tensile strength is also maintained.

In the case of the yarn 10 made of a polymer, ductility is increased by heating the yarn 10 to a specific temperature below the melting point of a raw material, but both ends of the yarn 10 are fixed inside the pressing device to be spread in a tight straight line structure. The yarn 10 spread in this way may be formed with the protrusions 100 by the rotation pressing of the rotary press while regular intervals are maintained between the protrusions 100 from the proximal end to the distal end of the yarn 10.

Since the yarn 10 is maintained in a tight straight line state with both ends thereof fixed, the yarn 10 may be processed by pressing in such a manner that the yarn 10 is heated and cooled (through quick heating and quick cooling) by the rotary press in a state in which a shrinkage deformation is suppressed. According to the pressing processing, the protrusions 100 which are compression-molded may be formed on the surface of the yarn 10 along the length of the yarn 10 in a structure in which the protrusions 100 are evenly distributed along the circumferential outer surface of the yarn 10.

Since the yarn 10 is made of a polymer material, the protrusions 100 may be formed on the surface of the yarn 10 while a molecular orientation is maintained.

A pressing space for the mold of the rotary press has a structure which is divided by a partition wall not exceeding 70 to 90 μm, and an interval between the pressing space and an outlet space of the mold may be in the range of 10 μm to 20 μm. When the interval between the pressing space and the outlet space is wider to exceed 20 μm, a problem may be caused in that a burr and a product are not separated from each other after press forming.

When a temperature is a room temperature, a compression-expansion phenomenon occurs during compression molding due to the elasticity of the polymer yarn 10. This means that, when the polymer yarn 10 is compression-molded at a room temperature by a strong force, a molding space is temporarily filled by a compression force, but when the compression force is removed, as a restoration expansion occurs to some extent, a burr portion between molds contracts and thus the shape of a product is formed differently from the shape of the molding space.

Wedge shapes with directionality, which are the shapes of ideal protrusions 100, may be formed around the surface of the yarn 10. Due to the feature that the protrusions 100 are formed by compressing the yarn 10 in a manner of heating and cooling the yarn 10, the protrusions 100 may be positioned in a structure in which they are staggered with each other.

The lifting thread (insertion thread) 1 made of a polymer may sufficiently secure a skin tissue holding force when performing a lifting function as the skin is pulled, and may also perform a function of promoting the regeneration of surrounding soft tissues.

The lifting thread (insertion thread) 1 made of a polymer may maintain the strength of the yarn 10 of the same size at 90% or more, since a cutting angle as in a method of cutting the surface of the yarn 10 is not formed and a physical knotting is not applied as well.

That is to say, because the lifting thread (insertion thread) 1 made of a polymer is manufactured by pressing type processing, the lifting thread 1 may have stronger traction and tensile forces although it is thinner in thickness, compared to a thread manufactured by the existing cutting method or other threads.

The lipidic tissue structures of the skin of most patients who visit, for example, plastic surgery hospitals for skin tightening procedures due to a phenomenon such as sagging or wrinkles of the skin may be classified depending on the degrees of hardness and density, and may be classified depending on the degrees of hardness and density into a weakest group, a weak group, a middle group and a higher group including a strong group.

As illustrated in FIGS. 1 and 2 for example, in the lifting thread (insertion thread) 1 in accordance with the first embodiment of the present disclosure, the protrusions 100 of a molded structure, which form the contour of splines, may be formed around the surface of the yarn 10.

The protrusions 100 may be formed in a regular or irregular zigzag structural arrangement in which the protrusions 100 maintain predetermined gaps while forming the contour of splines around the surface of the yarn 10. The protrusions 100 may be formed such that an engagement portion, which pulls the tissues of the skin in a process of lifting the skin after being inserted into the skin, has a structural shape, for example, as shown in FIG. 2, depending on the lipidic tissue state of the skin of a patient to be treated.

In other words, for example, as shown in FIG. 2, the protrusions 100 may be formed in the structure of a protruding shape for a hooking use applied to patients whose lipidic structures of the skin belong to a relatively weakest group among patients. In some cases, although not shown, the protrusions 100 may be formed in the structure of successive sawteeth which have ridges and grooves.

The protrusions 100 of the structure of successive sawteeth which have ridges and grooves are used to hook the tissues of the skin in patients whose lipidic structures of the skin belong to the weakest group. The sawteeth may properly exhibit the effect of a pulling force due to a hooking function according to the lifting thereof in a lipidic structure of the skin belonging to the weakest group.

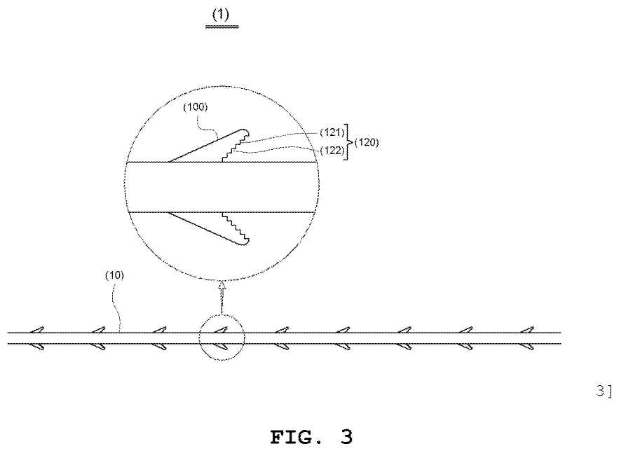

As illustrated in FIG. 3 for example, in a lifting thread (insertion thread) 1 in accordance with a second embodiment of the present disclosure, protrusions 100 of a molded structure, which form the contour of splines, may be formed around the surface of a yarn 10.

The protrusions 100 may be formed, through molding, in a structural arrangement in which the protrusions 100 maintain predetermined gaps while forming the contour of splines around the surface of the yarn 10. The protrusions 100 may be formed such that an engagement portion, which pulls the tissues of the skin in a process of lifting the skin after being inserted into the skin, has a structural shape, for example, as shown in FIG. 3, depending on the lipidic tissue state of the skin of a patient to be treated.

Namely, for example, as shown in FIG. 3, the protrusions 100 may be formed in a step-shaped structure 120 which has a successive structure of ridges 121 and grooves 122, for a hooking use applied to patients whose lipidic structures of the skin belong to a relatively weak group among patients.

The protrusions 100 including the step-shaped structure 120 which has the successive structure of the ridges 121 and the grooves 122 are used to hook the tissues of the skin in patients whose lipidic structures of the skin belong to the weak group. The step-shaped structure 120 may properly exhibit the effect of a pulling force due to a hooking function according to the lifting thereof in a lipidic structure of the skin belonging to the weak group.

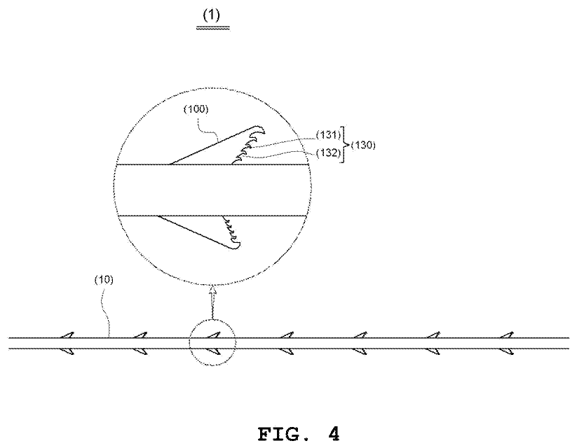

As illustrated in FIG. 4 for example, in a lifting thread (insertion thread) 1 in accordance with a third embodiment of the present disclosure, protrusions 100 of a molded structure, which form the contour of splines, may be formed around the surface of a yarn 10.

The protrusions 100 may be formed, through molding, in a structural arrangement in which the protrusions 100 maintain predetermined gaps while forming the contour of splines around the surface of the yarn 10. The protrusions 100 may be formed such that an engagement portion, which pulls the tissues of the skin in a process of lifting the skin after being inserted into the skin, has a structural shape, for example, as shown in FIG. 4, depending on the lipidic tissue state of the skin of a patient to be treated.

That is to say, for example, as shown in FIG. 4, the protrusions 100 may be formed in a hook-shaped structure 130 which has a successive structure of ridges 131 and grooves 132, for a hooking use applied to patients whose lipidic structures of the skin belong to a relatively middle group among patients.

The protrusions 100 including the hook-shaped structure 130 which has the successive structure of the ridges 131 and the grooves 132 are used to hook the tissues of the skin in patients whose lipidic structures of the skin belong to the middle group. The hook-shaped structure 130 may properly exhibit the effect of a pulling force due to a hooking function according to the lifting thereof in a lipidic structure of the skin belonging to the middle group.

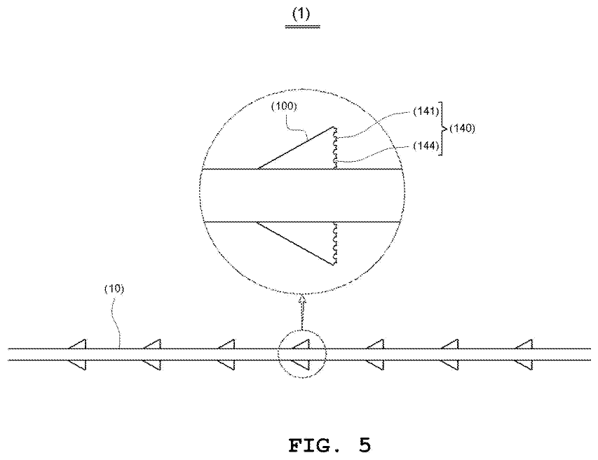

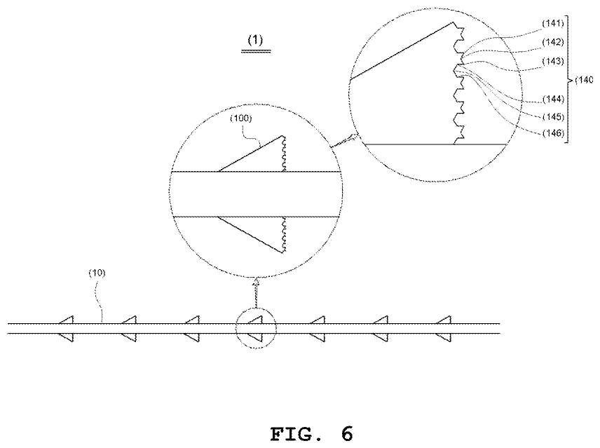

As illustrated in FIGS. 5 and 6 for example, in a lifting thread (insertion thread) 1 in accordance with a fourth embodiment of the present disclosure, protrusions 100 of a molded structure, which form the contour of splines, may be formed around the surface of a yarn 10.

The protrusions 100 may be formed, through molding, in a structural arrangement in which the protrusions 100 maintain predetermined gaps while forming the contour of splines around the surface of the yarn 10. The protrusions 100 may be formed such that an engagement portion, which pulls the tissues of the skin in a process of lifting the skin after being inserted into the skin, has a structural shape, for example, as shown in FIG. 5, depending on the lipidic tissue state of the skin of a patient to be treated.

In other words, for example, as shown in FIG. 5, the protrusions 100 may be formed in a polygon-shaped structure 140 which has a successive structure of ridges and grooves, for a hooking use applied to patients whose lipidic structures of the skin belong to a relatively strong group among patients.

The protrusions 100 including the polygon-shaped structure 140 which has the successive structure of the ridges and the grooves are used to hook the tissues of the skin in patients whose lipidic structures of the skin belong to a higher group including a strong group. The polygon-shaped structure 140 may properly exhibit the effect of a pulling force due to a hooking function according to the lifting thereof in a lipidic structure of the skin belonging to the higher group including the strong group.

Namely, for example, as shown in FIG. 6, each ridge of the polygon-shaped structure 140 may be formed to have a configuration including first and second pointed tips 141 and 143 which are symmetrical to each other and a third pointed tip 142 which is formed inversely to the first and second pointed tips 141 and 143, the first to third pointed tops 141 to 143 constituting one projecting mass.

In addition, each groove may be formed to have a configuration including first and second recessed tips 144 and 146 which are symmetrical to each other and a third recessed tip 145 which is formed inversely to the first and second recessed tips 144 and 146, the first to third recessed tips 144 to 146 constituting one depressed space.

The first and second pointed tips 141 and 142 and the third pointed tip 143 may act in a manner of strongly hooking a lipidic structure, belonging to the higher group including a strong group of the skin, in multiple directions. This is because first and second pointed tips 141 and 143 have a plurality of structures each of which projects as a single mass.

The first and second recessed tips 144 and 146 and the third recessed tip 145 may act in a manner of strongly biting a lipidic structure, belonging to the higher group including a strong group of the skin, in multiple directions. This is because first and second recessed tips 144 and 146 have a plurality of structures each of which is recessed as a single space.

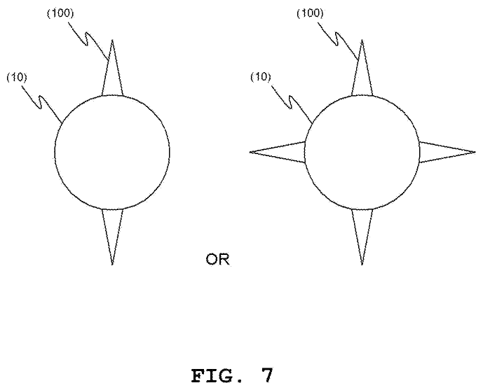

Meanwhile, in the lifting thread for a press according to the present disclosure, as shown in FIG. 7 for example, each two or each four of the protrusions 100 of a blade structure which are formed along the length of the yarn 10 may protrude on the section of the yarn 10. In some cases, an increased number of protrusions may protrude.

The protrusions 100 which protrude in the blade structure from the yarn 10 may of course protrude in an odd number, but it is preferred that the protrusions 100 protrude in an even number for strong pulling.

Besides, while there are a method by pressing and die cutting proposed in U.S. Patent No. 2006/005144 entitled “Tissue Holding Devices and Methods for Making the Same” and a method by injection molding referred to in the patent, these methods are ambiguous in the possibility of implementation of technology and are not commercially used by being commercialized at the present stage.

While various embodiments have been described above, it will be understood to those skilled in the art that the embodiments described are by way of example only. Accordingly, the present disclosure described herein should not be limited based on the described embodiments.

What is claimed is:

1. A lifting thread for a press, wherein the lifting thread is a thread in which protrusions of a protruding structure, evenly distributed along a surface portion of a yarn, are configured through a pressing method of a press device.

2. The lifting thread according to claim 1, wherein the protrusions are formed by a rotary press which presses a surface portion of the yarn at regular intervals while being rotated around a rear portion of the yarn spread in a predetermined straight length through an appropriate tension inside the press device.

3. The lifting thread according to claim 2, wherein the protrusions are formed in a pressing method through heating and cooling processing according to rotation and pressing operations of the rotary press.

4. The lifting thread according to claim 3, wherein the yarn is made of at least any one substance among PDO, PLLA and PCL.

5. The lifting thread according to claim 4, wherein a pressing space for a mold of the rotary press is 70 to 90 μm.
